(19) United States
(12) Patent Application Publication
(10) Pub. No.: US 2010/0137342 A1
(43) Pub. Date: Jun. 3, 2010

(54) METHODS FOR TREATING VASCULAR DISRUPTION DISORDERS

(75) Inventors: Mark Laughlin, Sunnyvale, CA (US); Thomas L. Thorne, Salt Lake City, UT (US)

Correspondence Address: Myriad PHARMACEUTICALS, Inc.;c/o CPA Global, P.O. Box 52050, Minneapolis, MN, 55402, US

(73) Assignee: Myriad Pharmaceuticals, Inc., Salt Lake City, UT (US)

(21) Appl. No.: 12/574,507

(22) Filed: Oct. 6, 2009

Related U.S. Application Data
(63) Continuation of application No. PCT/US2008/059914, filed on Apr. 10, 2008.
(60) Provisional application No. 60/910,939, filed on Apr. 10, 2007.

(51) Int. Cl. A61K 31/517; A61P 7/12

(52) U.S. Cl. 514/266.1

(57) ABSTRACT

Disclosed is (4-Methoxy-phenyl)-methyl-(2-methyl-quinazolin-4-yl)-amine hydrochloride effective as a vascular disrupting agent. (4-Methoxy-phenyl)-methyl-(2-methyl-quinazolin-4-yl)-amine hydrochloride is useful in the treatment of a variety of clinical conditions that are responsive to disruption of the vascular system, and in particular to its use in treating vascular macular degeneration.

### CROSS REFERENCE TO RELATED APPLICATIONS

This application is a continuation of International Application No. PCT/US2008/059914, filed Apr. 10, 2008 and published as WO 2008/124828, which claims the benefit of U.S. Provisional Application Ser. No. 60/910,939, filed on Apr. 10, 2007; both of which are incorporated by reference herein in their entirety.

### FIELD OF THE INVENTION

This invention is in the field of medicinal chemistry. In particular, the invention relates to (4-Methoxy-phenyl)-methyl-(2-methyl-quinazolin-4-yl)-amine hydrochloride as a vascular disrupting agent and use as treatment for diseases and disorders that are responsive to disruption of the vascular system.

### BACKGROUND OF THE INVENTION

The vascular system plays an important role in the growth and progression of various diseases and disorders. For example, vascular disrupting agents have been shown to have in vivo antitumor effects. See Liu et al., Cancer Chemother Pharmacol. 2007, 59(5):661-669. Epub 2006 Aug. 31; Shi and Siemann, Anticancer Res. 2005, 25(6B):3899-3904. The formation of new vasculature by angiogenesis is a key pathological feature of several diseases (J Folkman, New England Journal of Medicine 333, 1757-1763 (1995)). Neovascularisation is also a clinical feature of skin lesions in psoriasis, of the invasive pannus in the joints of rheumatoid arthritis patients and of atherosclerotic plaques. Retinal neovascularisation is pathological in macular degeneration and in diabetic retinopathy.

In all these diseases reversal of neovascularisation by damaging the vascular endothelium is expected to have a beneficial therapeutic effect. Furthermore, there remains a need for selective vascular disrupting agents that display improved potency, pharmacodynamic, and pharmacokinetic properties, such as oral bioavailability and duration of action, over already known compounds. Such compounds would prove to be useful for the treatment, prevention, or suppression of various pathologies that utilize or require the vascular system for growth.

### SUMMARY OF THE INVENTION

The present invention is related to the discovery that (4-Methoxy-phenyl)-methyl-(2-methyl-quinazolin-4-yl)-amine hydrochloride is active as a vascular disrupting agent. Accordingly, an aspect of the present invention is directed to the use of (4-Methoxy-phenyl)-methyl-(2-methyl-quinazolin-4-yl)-amine hydrochloride as therapy for diseases and disorders that are responsive to the disruption of vessels and/or vasculature system. Examples of diseases and disorders and/or conditions associated therewith that are responsive to vascular disruption include diabetic complications (including retinopathy and wound healing), psoriasis, rheumatoid arthritis, Kaposi's sarcoma, haemangioma, acute and chronic nephropathies, atheroma, arterial restenosis, autoimmune diseases, acute inflammation, excessive scar formation and adhesions, endometriosis, dysfunctional uterine bleeding, wound healing, and ocular diseases, including neovascularization of the retina, neovascularization of the choroid, neovascularization of ocular tumors, diabetic retinopathy, retinopathy of prematurity, retinoblastoma, neovascularization of the cornea, and macular degeneration. More specifically, ocular diseases include those which exhibit subfoveal choroidal neovascularization, including pathological myopia and exudative age-related macular degeneration. In specific embodiments, the disease or disorder is vascular macular degeneration (wet macular degeneration).

In one embodiment, (4-Methoxy-phenyl)-methyl-(2-methyl-quinazolin-4-yl)-amine hydrochloride is administered as therapy at a dose of not more than about 4.5 mg/m². For example, the invention provides a method for treatment at a dose of between about 0.3 to about 3.3 mg/m², such as between about 2.1 mg/m² and about 3.3 mg/m².

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

It is known that (4-Methoxy-phenyl)-methyl-(2-methyl-quinazolin-4-yl)-amine hydrochloride, is active as a potent tubulin inhibitor and cytotoxic agent. Here, it has been discovered that (4-Methoxy-phenyl)-methyl-(2-methyl-quinazolin-4-yl)-amine hydrochloride is a vascular disrupting agent.

The vascular system is important for the progression of many diseases and disorders. For example, neovascularisation is a clinical feature of skin lesions in psoriasis, of the invasive pannus in the joints of rheumatoid arthritis patients and of atherosclerotic plaques. Neovascularization of ocular tissue is a pathogenic condition characterized by vascular proliferation and occurs in a variety of ocular diseases with varying degrees of vision failure. The administration of (4-Methoxy-phenyl)-methyl-(2-methyl-quinazolin-4-yl)-amine hydrochloride for the pharmacological control of the neovascularization associated with non-malignant vascular proliferative disorders such as wet macular degeneration, proliferative diabetic retinopathy or retinopathy of prematurity would potentially benefit patients for which few therapeutic options are available.

In particular, the blood-retinal barrier (BRB) is composed of specialized nonfenestrated tightly-joined endothelial cells that form a transport barrier for certain substances between the retinal capillaries and the retinal tissue. The nascent vessels of the cornea and retina associated with the retinopathies are aberrant, much like the vessels associated with solid tumors. Tubulin binding agents, inhibitors of tubulin polymerization and vascular targeting agents, may be able to attack the aberrant vessels because these vessels do not share architectural similarities with the blood retinal barrier. Tubulin binding agents may halt the progression of the disease much like they do with a tumor-vasculature.

For example, ocular indications treatable by the administration of (4-Methoxy-phenyl)-methyl-(2-methyl-quinazolin-4-yl)-amine hydrochloride include non-malignant vascular proliferative diseases characterized by corneal, iris, trabecular meshwork, retinal, subretinal, optical nerve head, or choroidal neovascularization.

Corneal neovascularization occurs in the following: trachoma (Chlamydia trachomatis), viral interstitial keratitis, microbial keratoconjunctivitis, corneal transplantation and burns. It may be caused by infection (trachoma, herpes, leishmaniasis, onchoceroiasis), transplantation, burns (heat, alkalai), trauma, nutritional deficiency and contact lens induced damage. Diseases involving iris neovascularization include rubeosis iritis, Fuchs' heteochromic iridocyclitis, and developmental hypoplasia of the iris.

Retinal and/or choroidal neovascularization occurs in macular degeneration, diabetic retinopathy, sickle cell retinopathy, and retinopathy of prematurity. Choroidal neovascularization occurs when vessels from the choroidal membrane grow through a break in Bruch's membrane and into the subretinal pigment epithelium or the subretinal space, manifesting as fluid accumulation (edema) and or hemorrhaging. This in itself can lead to severe vision loss, however the retinal pigment epithelium or the neurosensory retina may also detach. In a particular embodiment, the invention involves the treatment of highly proliferative subfoveal choroidal neovascularization which occurs as a result of or concurrent with exudative (wet) forms of age-related macular degeneration, diabetic retinopathy, retinopathy of prematurity, pathologic myopia, posterior uveitis, chronic uveitis, ocular histoplasmosis syndrome, macular edema, retinal vein occlusion, angiod streaks, choroidal rupture, multifocal choroiditis, ischemic retinal disease, and other uveitic entities.

A particular example of subfoveal choroidal neovascularization occurs as a result of or concurrent with pathological myopia. High myopia (extreme near sightedness) is a condition in characterized by abnormal growth of the eyeball causing stretching of the retina and Bruch's membrane. A gradual decrease in vision occurs when the macula is thinned as a result of the retinal stretching. The thinning of Bruch's membrane can result in cracks through which neovasculature can grow from the choroid underneath the retina.

Subfovial choroidal neovascularization can cause sudden and severe loss of vision. Another particular example of subfoveal choroidal neovascularization occurs as a result of, or concurrent with, exudative age-related macular degeneration. Anterior chamber neovascularization occurs in neovascular glaucoma.

Another disease that may be treated with (4-Methoxy-phenyl)-methyl-(2-methyl-quinazolin-4-yl)-amine hydrochloride is vascular macular degeneration (wet macular degeneration). The wet-form of macular degeneration is a rapidly progressing disease that almost always results in severe vision loss. Vision-loss associated with Wet macular degeneration is the result of sub-retinal neovascularization. The rapid growth of the sub-retinal blood vessels causes the overlying layer of retinal cells to buckle and become detached from the nutrient-rich choroid. In extreme cases of Wet macular degeneration the proliferating vessels penetrate the retina and infiltrate the vitreous humor.

Another debilitating ocular disease is Retinopathy of Prematurity (ROP). ROP is an eye disease that occurs in a significant percentage of premature babies. The last 12 weeks of a full-term delivery (weeks 28 to 40) are particularly active months in the development of the fetal eye. The pre-natal development of the retinal blood supply (choroid) initiates at the optic nerve on week 16 and progresses in a radial fashion towards the anterior region of the retina until birth (week 40). If birth is premature, the retinal vasculature does not have enough time to fully develop and the anterior edges of the retina become deprived of oxygen. The lack of anterior-retinal oxygenation is the underlying cause of ROP. (See, The Association of Retinopathy of Prematurity and Related Diseases, Franklin, Mich.)

In premature infants a significant portion of the anterior retina is deprived of an adequate blood supply. The oxygen deprived anterior retina responds by signaling for the growth of new vessels. Abnormal neovascularization in the zone between the anterior and posterior retina initiates a cascade of events with severe pathologic consequences. As new vessels grow in response to the chemical signals, arterio-venous shunts are formed in the zone between vascularized posterior retina and the avascular anterior retina. These vascular shunts gradually enlarge, becoming thicker and more elevated. The new vessels are accompanied by infiltrating fibroblasts, which produce fibrous scar tissue. Eventually, a ring of scar tissue is formed which is attached to the retina as well as to the vitreous gel. The ring of scar tissue may extend for 360 degrees around the inside of the eye. When this scar tissue contracts it pulls the retina and produces a retinal detachment. If enough scar tissue forms, the retina can become completely detached. Premature neonates are at risk for developing ROP because they have been taken out of the protective environment of the uterus and are exposed to a variety of angiogenic stimuli, including medications, high levels of oxygen, and variations in light and temperature. Some or all of these factors may have an effect on the development of ROP. Fortunately, most premature infants do not develop ROP, and most infants with ROP improve spontaneously. If, ROP does develop, it usually occurs between 34 and 40 weeks after conception, regardless of gestational age at birth.

Another debilitating ocular disease occurs in patients who suffer from diabetes mellitus. Approximately 14 million Americans have diabetes mellitus. In addition to causing numerous systemic complications (such as kidney failure, hypertension, and cardiovascular disease), diabetes is one of the leading causes of blindness among working-age Americans.

In fact, the risk of blindness to persons with diabetes is 25 times greater than that of the general population. Many patients with diabetic eye problems are asymptomatic despite the presence of vision-threatening disease. If diabetic eye disease is left untreated, it can lead to serious visual loss. Decreased vision due to diabetes can be caused by several mechanisms, and treatment needs to be tailored to the individual's needs. (See, The Center for Disease Control, “The Prevention and Treatment of Diabetes Mellitus-A Guide for Primary Care Practitioners”). Many diabetics notice blurred vision when their blood sugar is particularly high or low. This blurred vision results from changes in the shape of the lens of the eyes, and usually reverse when their blood sugar returns to normal. Diabetes is a disease that affects not only the patient's blood sugar levels, but also the blood vessels. Symptoms associated with diabetes (including elevated blood pressure) cause damage to the microcirculatory system including the capillaries associated with the retina. Capillary damage results in a decreased flow of blood to isolated regions of the retina. In addition, the damaged blood vessels tend to leak, which produces swelling within the retina.

There are two main categories of diabetic eye disease. The first category is termed background diabetic retinopathy or non-proliferative retinopathy. This is essentially the earliest stage of diabetic retinopathy. This stage is characterized by damage to small retinal blood vessels which results in the effusion of fluid (blood) into the retina. Most visual loss during this stage is due to the fluid accumulating in the macula. This accumulation of fluid is called macular edema, and can cause temporary or permanent decreased vision. The second category of diabetic retinopathy is termed proliferative diabetic retinopathy. Proliferative retinopathy is the end result of diabetes-induced damage sustained by the retinal capillary bed (choroid). Damage to the choroid causes oxygen deprivation in the retina. The retinal tissue responds to its anoxic environment by producing angiogenic cytokines that stimulate neovascularization. As was previously stated, neovascularization of the retina causes bleeding in the eye, retinal scar tissue, retinal detachments, and any of one of these symptoms can cause decreased vision or blindness. Diabetics often also suffer from neovascular glaucoma, which manifests in rubeosis, blood vessels growing on the iris that causes closure of the angle.

Diabetic retinopathy can occur in both Type I diabetics (onset of diabetes prior to age 40) and Type II diabetics (onset after age 40), although it tends to be more common and more severe in Type I patients. Because Type II diabetes is often not diagnosed until the patient has had the disease for many years, diabetic retinopathy may be present in a Type II patient at the time diabetes is discovered.

The treatment of diabetic retinopathy depends upon multiple factors, including the type and degree of retinopathy, associated ocular factors such as cataract or vitreous hemorrhage, and the medical history of the patient. Treatment options include options such as laser photocoagulation, cryotherapy (freezing), and vitrectomy surgery. Blindness due to diabetic retinopathy is preventable in most cases.

Accordingly, in one embodiment of the invention, (4-Methoxy-phenyl)-methyl-(2-methyl-quinazolin-4-yl)-amine hydrochloride is administered as therapy for diseases and disorders that are responsive to the disruption of the vasculature system. In specific embodiments, (4-Methoxy-phenyl)-methyl-(2-methyl-quinazolin-4-yl)-amine hydrochloride is administered as therapy for diabetic complications (including retinopathy and wound healing), psoriasis, rheumatoid arthritis, Kaposi's sarcoma, haemangioma, acute and chronic nephropathies, atheroma, arterial restenosis, autoimmune diseases, acute inflammation, excessive scar formation and adhesions, endometriosis, dysfunctional uterine bleeding, wound healing, and ocular diseases, including neovascularization of the retina, neovascularization of the choroid, neovascularization of ocular tumors, diabetic retinopathy, retinopathy of prematurity, retinoblastoma, neovascularization of the cornea, and macular degeneration. More specifically, ocular diseases include those which exhibit subfoveal choroidal neovascularization, including pathological myopia and exudative age-related macular degeneration. In on embodiment, (4-Methoxy-phenyl)-methyl-(2-methyl-quinazolin-4-yl)-amine hydrochloride is administered as therapy for wet macular degeneration.

In certain embodiments, (4-Methoxy-phenyl)-methyl-(2-methyl-quinazolin-4-yl)-amine hydrochloride is administered as therapy at a dose of not more than about 4.5 mg/m², such as not more than about 3.3 mg/m², not more than about 2.7 mg/m², and not more than about 2.1 mg/m². For example, the invention provides a method for treatment at a dose of between about 0.3 to about 3.3 mg/m², such as between about 2.1 mg/m² and about 3.3 mg/m². In some embodiments (4-Methoxy-phenyl)-methyl-(2-methyl-quinazolin-4-yl)-amine hydrochloride may be administered at a dose of not more than about 2.5 mg/m², such as not more than about 1.5 mg/m², or not more than about 0.5 mg/m². In certain embodiments (4-Methoxy-phenyl)-methyl-(2-methyl-quinazolin-4-yl)-amine hydrochloride may be administered at a dose of about 2.1 mg/m², about 2.7 mg/m², or about 3.3 mg/m².

In certain embodiments, psoriasis, rheumatoid arthritis, Kaposi's sarcoma, haemangioma, acute and chronic nephropathies, atheroma, arterial restenosis, autoimmune diseases, acute inflammation, excessive scar formation and adhesions, endometriosis, dysfunctional uterine bleeding and/or ocular diseases are treated with (4-Methoxy-phenyl)-methyl-(2-methyl-quinazolin-4-yl)-amine hydrochloride at a dose of not more than about 4.5 mg/m², such as not more than 3.3 mg/m², or not more than about 2.1 mg/m². In particular embodiments, wet macular degeneration is treated with (4-Methoxy-phenyl)-methyl-(2-methyl-quinazolin-4-yl)-amine hydrochloride at a dose of not more than about 4.5 mg/m², such as not more than 3.3 mg/m², or not more than about 2.1 mg/m².

In practicing the present invention, (4-Methoxy-phenyl)-methyl-(2-methyl-quinazolin-4-yl)-amine hydrochloride may be prepared using methods known to those skilled in the art. Specifically, (4-Methoxy-phenyl)-methyl-(2-methyl-quinazolin-4-yl)-amine hydrochloride may be prepared according to International Pat. Publication No. WO 2005/003100 and as illustrated by the exemplary reaction in Scheme 1.

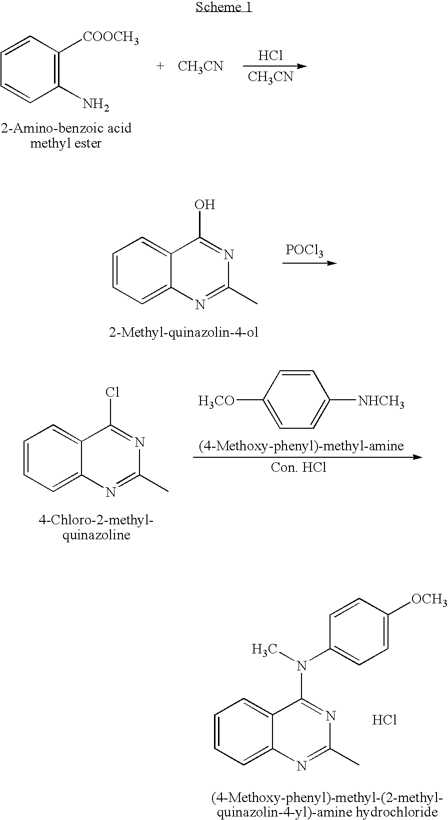

The therapeutic methods of present invention also include methods comprising administering to an animal an effective amount of a compound, or a pharmaceutically acceptable salt, acid or base of (4-Methoxy-phenyl)-methyl-(2-methyl-quinazolin-4-yl)-amine hydrochloride. In one embodiment, a pharmaceutical composition comprising (4-Methoxy-phenyl)-methyl-(2-methyl-quinazolin-4-yl)-amine hydrochloride, or a pharmaceutically acceptable salt, acid, or base of said compound, in combination with a pharmaceutically acceptable vehicle is administered. Examples of pharmaceutically acceptable addition salts for (4-Methoxy-phenyl)-methyl-(2-methyl-quinazolin-4-yl)-amine hydrochloride, (or base thereof) include inorganic and organic acid addition salts, such as hydrochloride, hydrobromide, phosphate, sulphate, citrate, lactate, tartrate, maleate, fumarate, mandelate and oxalate; and inorganic and organic base addition salts with bases, such as sodium hydroxy, Tris(hydroxymethyl)aminomethane (TRIS, tromethane) and N-methyl-glucamine. The present invention also includes methods comprising administering to an animal an effective amount of (4-Methoxy-phenyl)-methyl-(2-methyl-quinazolin-4-yl)-amine hydrochloride, or a pharmaceutically acceptable salt or prodrug thereof, and one or more liquid diluents. Such compositions include compositions disclosed in PCT Pub. No. WO 2006/138608, and may be manufactured according to the methods disclosed therein, the relevant portions of which are incorporated herein by reference.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the compounds of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired. Furthermore, local (non-systemic) delivery of agents to the eye can be achieved using intravitreal injection, sub-Tenon's injection, ophthalmic drops iontophoresis, and implants and/or inserts. Systemic administration may be accomplished by administration of the agents into the bloodstream at a site which is separated by a measurable distance from the diseased or affected organ or tissue, in this case they eye. In certain embodiments, modes of systemic administration include parenteral or oral administration.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### Example 1

### Preparation of (4-Methoxy-phenyl)-methyl-(2-methyl-quinazolin-4-yl)-amine hydrochloride

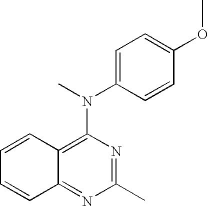

### (4-Methoxy-phenyl)-methyl-(2-methyl-quinazolin-4-yl)-amine hydrochloride

a) 4-Chloro-2-methyl-quinazoline: A stirred suspension of 2-methyl-4(3H)-quinazolinone (5 g, 31.2 mmol) in POCl₃ (100 mL) was heated at 120° C. for 3 h. The excess POCl₃ was removed under vacuum, then to the residue was added crushed ice and 200 mL of saturated NaHCO₃, and the mixture was extracted with ethyl acetate (200 mL×2). The combined extracts were washed with water, saturated NaCl, dried over anhydrous MgSO₄, filtered and concentrated. The crude product was purified by column chromatography (5-8% ethyl acetate/hexane) to give the title compound (2.5 g, 14.0 mmol, 45%). ¹H NMR (CDCl₃): 8.21-8.25 (m, 1H), 7.89-7.99 (m, 2H), 7.66 (ddd, 1H, J=1.8, 6.6, 8.7), 2.87 (s, 3H).

b) (4-Methoxy-phenyl)-methyl-(2-methyl-quinazolin-4-yl)-amine hydrochloride: The title compound was prepared from 4-chloro-2-methyl-quinazoline (2.31 g, 12.9 mmol) and (4-methoxy phenyl)-methyl-amine (2.0 g, 14.6 mmol) by a procedure similar to example 1b and was isolated as solids (2.90 g, 9.18 mmol, 71%). ¹H NMR (CDCl₃): 8.53 (dd, 1H, J=0.6, 8.1), 7.7 (ddd, 1H, J=1.2, 7.2, 8.4), 7.22 (m, 2H), 7.13 (ddd, 1H, J=1.2, 7.2, 8.7), 7.05 (m, 2H), 6.76 (d, 1H, J=8.7), 3.91 (s, 3H), 3.78 (s, 3H), 2.96 (s, 3H).

### Example 2

### Pharmaceutical Composition

A pharmaceutical composition is prepared by combining and mixing 100 grams of (4-Methoxy-phenyl)-methyl-(2-methyl-quinazolin-4-yl)-amine hydrochloride and 1 gram of BHT and dissolving into 10 liters of D5W with the pH adjusted to pH=5 with hydrochloric acid. This solution is sterile filtered using a 0.2 μm Teflon filter (PTFE).

### Example 3

### Pharmaceutical Composition

A pharmaceutical composition was formed by dissolving 300.1 grams (4-Methoxy-phenyl)-methyl-(2-methyl-quinazolin-4-yl)-amine hydrochloride into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 4

### Pharmaceutical Composition

A pharmaceutical composition was formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 5

### Pharmaceutical Composition

A pharmaceutical composition is formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 11.652 kg viscosity reducing agent (ethanol 190 proof), and 2 kg WFI (water for injection). This solution is sterile filtered through a 0.2 μm Millipore Durapore filter (PVDF), and packaged into 10 ml sterile glass vials.

### Example 6

### Method of Administration

About 0.01 ml to about 50 ml of the pharmaceutical composition of Example 5 is accurately measured and then added to an i.v. bag containing about 100 ml to about 1000 ml of sterile dextrose 5% in water (D5W). The amount of pharmaceutical composition and D5W used varies according to the desired therapeutic dose and size of the patient. The resulting mixture is then parenterally infused into the patient.

### Example 7

### Vascular Disruption Effects of (4-Methoxy-phenyl)-methyl-(2-methyl-quinazolin-4-yl)-amine hydrochloride

(4-Methoxy-phenyl)-methyl-(2-methyl-quinazolin-4-yl)-amine hydrochloride was tested in a human tumor xenograft model to determine its vascular disruption effects. Crl:Nu/Nu-nuBR mice were grown and injected with 1×107 human ovarian OVCAR-3 carcinoma cells on the right flank and allowed to grow to various tumor volumes. Mice were then dosed with either vehicle, 100 mg/kg combretastatin A-4 phosphate (CA4P) (obtained from Toronto Research Chemicals, Ontario, Canada) or 10 mg/kg (4-Methoxy-phenyl)-methyl-(2-methyl-quinazolin-4-yl)-amine hydrochloride.

Animals were housed by groups in Positive Individual Ventilation (PIV) cages in flat-bottom cages (Thoren Caging Systems, Hazelton, Pa.) with no more than ten mice per cage. Cages contained autoclaved TEK-Fresh bedding (Harlan, Indianapolis, Ind.), which was changed every seven days. Environmental controls were set to maintain a temperature between 65° F. and 75° F. with a relative humidity of 30-70% in a 12:12 hour light:dark cycle. Animals were feed gamma-irradiated 2019 rodent chow ad libitum (Harlan, Indianapolis, Ind.). Tap water was sterilized using manufacture recommended conditions and supplied via an automated watering system ad libitum (Edstrom Industries, Waterford, Wis.). Twenty four hours after dosing, mice were sacrificed and tumors and hearts removed, fixed, sectioned and stained with hematoxylin and eosin Y (each obtained from Richard Allen Scientific, Kalamazoo, Mich.).

Examination of all tumors revealed that necrosis was a prominent feature. Additionally, the tumors were highly anaplastic with marked pleomorphism and high mitotic indices. Within vehicle or compound treated cohorts, there was little variation in tumor morphology from animal to animal. This tumor was naturally arranged in packets surrounded by a thin fibrous stroma.

Rapid tumor proliferation often results in central necrosis of individual packets because the tumor outgrows the vascular supply due to rapid proliferation. In the vehicle control, this process appeared to be responsible for the central necrosis observed in some tumor nodules. Also, tumor tissue from the vehicle control contained large cystic areas suggestive of germinal follicles.

In tumors from mice treated with either (4-Methoxy-phenyl)-methyl-(2-methyl-quinazolin-4-yl)-amine hydrochloride or CA4P, the necrosis appeared to originate from a different process than rapid proliferation. Instead of necrosis of individual tumor nodules, the necrosis was widespread and involved the supporting stroma, as well as the neoplasm. Blood vessels in the necrotic areas were consistently congested with perivascular hemorrhage. This pattern of necrosis suggests that the blood supply to the tumors was disrupted at some point. Unlike the vehicle control, the central area of the tumors treated with either (4-Methoxy-phenyl)-methyl-(2-methyl-quinazolin-4-yl)-amine hydrochloride or CA4P had extensive necrosis with a rim of viable tumor tissue around the periphery. Overall, the degree of necrosis appeared greater in the (4-Methoxy-phenyl)-methyl-(2-methyl-quinazolin-4-yl)-amine hydrochloride treated animals than in the CA4P treated animals.

### Example 8

### Phase I Clinical Trial of Administration of (4-Methoxy-phenyl)-methyl-(2-methyl-quinazolin-4-yl)-amine hydrochloride for Subjects with Refractory Solid Tumors

An open-label, dose-escalating, multiple-dose study to define the safety, tolerability and phamacokinetics of weekly intravenous administration of (4-Methoxy-phenyl)-methyl-(2-methyl-quinazolin-4-yl)-amine hydrochloride was performed. A dosing schedule (each 4 week cycle) was performed for (4-Methoxy-phenyl)-methyl-(2-methyl-quinazolin-4-yl)-amine hydrochloride weekly for 3 weeks with no infusion on the fourth week of each cycle. Subjects with refractory solid tumors were enrolled in cohorts of 3. During Cycle 1, subjects were hospitalized during each infusion of (4-Methoxy-phenyl)-methyl-(2-methyl-quinazolin-4-yl)-amine hydrochloride and remained for observation and safety evaluation for approximately 24 hours following the end of the infusion. All subject had continuous telemetry for 2 hours prior to infusion, for 1-2 hour infusion and for 3 hours after the end of the infusion. Any clinically significant electrocardiographic (ECG) wave form abnormality was recorded and prolongation of the monitoring period extended at the discretion of the principal investigator.

Electrocardiograms were obtained prior to starting the infusion and within 30 minutes of the end of infusion for each infusino of the first cycle. Electrocardiograms on Day 1 were obtained in triplicate 5 minutes apart.

Neurocognitive assessments were made by administration of the Mini-Mental State Examination (MMSE), the Hopkins Verbal Learning and timed Grooved Pegboard tests before administration of the intravenous infusion and approximately 24 hours of the infusion at each weekly administration of the first cycle.

On days 1, 8, and 15 of each cycle, vital signs were obtained prior to the first dose, at 15, 30, and 60 minutes after the initiation of the infusion, and at 0.5, 1, 1.5, 2, and 4 hours after the end of the intravenous infusion. Vital signs at all time points beyond the start of the intravenous infusion included heart rate, blood pressure and respirations. Temperature ws measured at the end of the infusion and 4 hours later.

Individual subjects were allowed to continue on repeated weekly×3 administrations every 28 days with no dose increase provided there was no unacceptable toxicity or disease progression.

Tumor response was evaluated by response evaluation criteria in solid tumors (RECIST) criteria. To prevent sever hypersensitivity reactions due to Cremophor® EL, subjects were premedicated with oral dexamethasone (20 mg) administered approximately 12 and 6 hours before the intravenous infusion with (4-Methoxy-phenyl)-methyl-(2-methyl-quinazolin-4-yl)-amine hydrochloride, diphenhydramine (50 mg) or its equivalent administered intravenously 30-60 minutes before (4-Methoxy-phenyl)-methyl-(2-methyl-quinazolin-4-yl)-amine hydrochloride, and cimetidine (300 mg) or ranitidine (50 mg) administered intravenously 30-60 minutes before (4-Methoxy-phenyl)-methyl-(2-methyl-quinazolin-4-yl)-amine hydrochloride.

Dose excalation of subjects proceeded sequentially as presented in Table 1 below:

TABLE 1

| Cohort Number | Dose level (modified Fibonacci series) |
|---|---|
| Cohort 1 | Dose 1 = 0.3 mg/m² |
| Cohort 2 | Dose 2 = 0.6 mg/m² |
| Cohort 3 | Dose 3 = 1.0 mg/m² |
| Cohort 4 | Dose 4 = 1.5 mg/m² |
| Cohort 5 | Dose 5 = 2.1 mg/m² |
| Cohort 6 | Dose 6 = 2.7 mg/m² |
| Cohort 7 | Dose 7 = 3.3 mg/m² |

The results of the Phase 1 Trial show that there is no evidence of cytotoxity peripherally at the administered doses. There were incidences of intratumor bleeding and the dose limiting toxicity was demonstrated to be vascular in nature, manifested by an acute coronary syndrome. There were no significant effects on cardiac conduction (PR, QRS or QTc) but there was a dose-related increase in systolic blood pressure and occasional episodes of bradycardia. Accordingly, (4-Methoxy-phenyl)-methyl-(2-methyl-quinazolin-4-yl)-amine hydrochloride is thus shown to be safe and tolerable.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of treating diseases and disorders responsive to vascular disruption in a mammal in need of such treatment, comprising administering to the mammal an effective amount of (4-Methoxy-phenyl)-methyl-(2-methyl-quinazolin-4-yl)-amine, or a pharmaceutically acceptable salt thereof.

2. The method of claim 1, wherein the diseases and disorders are chosen from psoriasis, rheumatoid arthritis, Kaposi's sarcoma, haemangioma, acute and chronic nephropathies, atheroma, arterial restenosis, autoimmune diseases, acute inflammation, excessive scar formation and adhesions, endometriosis, dysfunctional uterine bleeding and ocular diseases.

3. The method of claim 1, wherein the disease or disorder is vascular macular degeneration.

4. The method of claim 1, wherein the pharmaceutically acceptable salt is (4-Methoxy-phenyl)-methyl-(2-methyl-quinazolin-4-yl)-amine hydrochloride.

5. The method of claim 4, wherein the effective amount of (4-Methoxy-phenyl)-methyl-(2-methyl-quinazolin-4-yl)-amine hydrochloride is administered at a dose of not more than about 4.5 mg/m².

6. The method of claim 4, wherein the effective amount of (4-Methoxy-phenyl)-methyl-(2-methyl-quinazolin-4-yl)-amine hydrochloride is administered at a dose of not more than about 3.3 mg/m².

7. The method of claim 4, wherein the effective amount of (4-Methoxy-phenyl)-methyl-(2-methyl-quinazolin-4-yl)-amine hydrochloride is administered at a dose of not more than about 2.7 mg/m².

8. The method of claim 4, wherein the effective amount of (4-Methoxy-phenyl)-methyl-(2-methyl-quinazolin-4-yl)-amine hydrochloride is administered at a dose of not more than about 2.2 mg/m².

9. The method of claim 4, wherein the effective amount of (4-Methoxy-phenyl)-methyl-(2-methyl-quinazolin-4-yl)-amine hydrochloride is administered at a dose of between about 2.1 mg/m² and about 3.3 mg/m².

10. A method of treating neovascularization in a mammal in need of such treatment, comprising administering to the mammal an effective amount of (4-Methoxy-phenyl)-methyl-(2-methyl-quinazolin-4-yl)-amine, or a pharmaceutically acceptable salt thereof.

11. The method of claim 10, wherein the neovascularization is a result of skin lesions in psoriasis, invasive pannus in the joints due to rheumatoid arthritis, or atherosclerotic plaques.

12. The method of claim 10, wherein the neovascularization is associated with wet macular degeneration, proliferative diabetic retinopathy, or retinopathy of prematurity.

13. The method of claim 10, wherein the neovascularization is corneal neovascularization, iris neovascularization, trabecular meshwork neovascularization, retinal neovascularization, subretinal neovascularization, optical nerve head neovascularization, or choroidal neovascularization.

14. The method of claim 1, wherein the pharmaceutically acceptable salt is (4-Methoxy-phenyl)-methyl-(2-methyl-quinazolin-4-yl)-amine hydrochloride.

15. The method of claim 14, wherein the effective amount of (4-Methoxy-phenyl)-methyl-(2-methyl-quinazolin-4-yl)-amine hydrochloride is administered at a dose of between about 2.1 mg/m² and about 3.3 mg/m².

16. A method of treating diabetic eye disease in a mammal in need of such treatment, comprising administering to the mammal an effective amount of (4-Methoxy-phenyl)-methyl-(2-methyl-quinazolin-4-yl)-amine, or a pharmaceutically acceptable salt thereof.

17. The method of claim 16, wherein the pharmaceutically acceptable salt is (4-Methoxy-phenyl)-methyl-(2-methyl-quinazolin-4-yl)-amine hydrochloride.

18. The method of claim 17, wherein the effective amount of (4-Methoxy-phenyl)-methyl-(2-methyl-quinazolin-4-yl)-amine hydrochloride is administered at a dose of between about 2.1 mg/m² and about 3.3 mg/m².

19. The method of claim 16, wherein the effective amount of (4-Methoxy-phenyl)-methyl-(2-methyl-quinazolin-4-yl)-amine hydrochloride is administered at a dose of not more than about 4.5 mg/m².

20. The method of claim 16, wherein the diabetic eye disease comprises diabetic retinopathy.
